(19) United States
(12) Patent Application Publication
(10) Pub. No.: US 2021/0369503 A1
(43) Pub. Date: Dec. 2, 2021

(54) MEDICAL NEGATIVE PRESSURE LAMINATION COMPONENT

(71) Applicant: Microjet Technology Co., Ltd., Hsinchu (TW)

(72) Inventors: Hao-Jan Mou, Hsinchu (TW); Chung-Wei Kao, Hsinchu (TW); Jia-Yu Liao, Hsinchu (TW); Hung-Hsin Liao, Hsinchu (TW); Chi-Feng Huang, Hsinchu (TW); Yung-Lung Han, Hsinchu (TW)

(73) Assignee: Microjet Technology Co., Ltd., Hsinchu (TW)

(21) Appl. No.: 17/325,692

(22) Filed: May 20, 2021

(30) Foreign Application Priority Data
May 29, 2020 (TW) 109118184

(51) Int. Cl. A61F 13/02; A61M 1/00

(52) U.S. Cl. CPC A61F 13/0216; A61M 1/962; A61M 1/964; A61M 2205/0294; A61M 2205/3331; A61M 2205/3327; A61M 2205/8206

(57) ABSTRACT

The present disclosure provides a medical negative pressure lamination component including an airtight patch, a dressing patch, a battery module, a slim-type pump and a sensing and controlling module. The airtight patch includes a communication portion and a dressing area in fluid communication with each other. The dressing patch is accommodated in the dressing area. The slim-type pump is electrically connected to the battery module. The sensing and controlling module is electrically connected to the battery module and the slim-type pump, and detects and controls a gas pressure and a gas flow provided by the slim-type pump. The dressing patch is attached on the skin surface, and is covered and accommodated by the airtight patch. When the slim-type pump is actuated, the air between the airtight patch and the skin surface is drawn out by the slim-type pump through the communication portion, and a negative pressure is formed therebetween.

### FIELD OF THE INVENTION

The present disclosure relates to a medical negative pressure lamination component, and more particularly to a medical negative pressure lamination component capable of generating a negative pressure between an airtight patch and a skin surface of a user by a pump, so that the dressing patch can be tightly attached on the skin surface of the user.

### BACKGROUND OF THE INVENTION

When a conventional medical patch is used, inconvenience frequently occurs due to the diversity of the skin types of users. Owing to the difference between the skin types, the adhesion strength of the glue layer of the patch usually changes with respect to the different skins. If the adhesion strength of the glue layer of the patch is too strong, the patch is hard to be removed, and/or partial of the glue layer is remained on the skin of the user when the patch is removed. If the adhesion strength of the glue layer of the patch is insufficient, the patch is easily detached from the skin.

Therefore, there is a need of providing a medical negative pressure lamination component so as to obviate the drawbacks encountered from the prior arts, so that the medical patch can be tightly attached on the skin surface through the negative pressure.

### SUMMARY OF THE INVENTION

It is an object of the present disclosure to provide a medical negative pressure lamination component. By drawing out the gas between the airtight patch and the skin surface of a user by a slim-type pump, the negative pressure is formed therebetween. As a result, the airtight patch is tightly attached on the skin surface.

In accordance with an aspect of the present disclosure, there is provided a medical negative pressure lamination component. The medical negative pressure lamination component includes an airtight patch, a dressing patch, a battery module, a slim-type pump and a sensing and controlling module. The airtight patch includes a communication portion and a dressing area. The communication portion and the dressing area are in fluid communication with each other. The dressing patch is accommodated in the dressing area. The slim-type pump is electrically connected to the battery module. The sensing and controlling module is electrically connected to the battery module and the slim-type pump, and detects and controls a gas pressure and a gas flow provided by the slim-type pump. The dressing patch is attached on the skin surface. The airtight patch covers on the dressing patch and accommodates the dressing patch. When the slim-type pump is actuated, the air between the airtight patch and the skin surface is drawn out by the slim-type pump through the communication portion, and a negative pressure is formed between the airtight patch and the skin surface, so that the airtight patch and the dressing patch are tightly attached on the skin surface.

### BRIEF DESCRIPTION OF THE DRAWINGS

The above contents of the present invention will become more readily apparent to those ordinarily skilled in the art after reviewing the following detailed description and accompanying drawings, in which:

FIG. 1A is a schematic perspective view illustrating the medical negative pressure lamination component of the present disclosure;

FIG. 1B is an exploded view illustrating the medical negative pressure lamination component of the present disclosure;

FIG. 2 is a schematic cross-sectional view illustrating the sensing and controlling module of the present disclosure;

FIG. 3A is a schematic perspective view illustrating the slim-type pump of the present disclosure;

FIG. 3B is an exploded view illustrating the slim-type pump of the present disclosure;

FIG. 4A is a schematic perspective view illustrating the base plate of the present disclosure;

FIG. 4B is a bottom view illustrating the base plate of the present disclosure;

FIG. 5A is an exploded view illustrating the gas pump of the present disclosure;

FIG. 5B is another exploded view illustrating the gas pump of the present disclosure and taken from a different perspective;

FIG. 6A is a schematic cross-sectional view illustrating the gas pump of the present disclosure;

FIGS. 6B to 6D schematically illustrate the actions of the gas pump of the present disclosure;

FIGS. 7A and 7B schematically illustrate the air flow of the slim-type pump of the present disclosure; and

FIG. 7C schematically illustrates the structure of the slim-type pump of the present disclosure which avoids the gas reflowing.

### DETAILED DESCRIPTION OF THE PREFERRED EMBODIMENT

The present invention will now be described more specifically with reference to the following embodiments. It is to be noted that the following descriptions of preferred embodiments of this invention are presented herein for purpose of illustration and description only. It is not intended to be exhaustive or to be limited to the precise form disclosed.

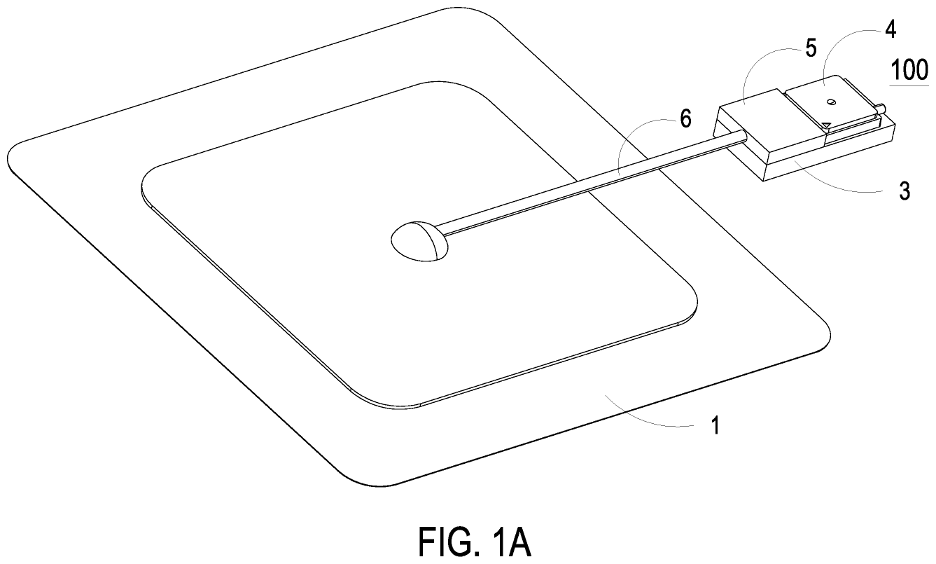

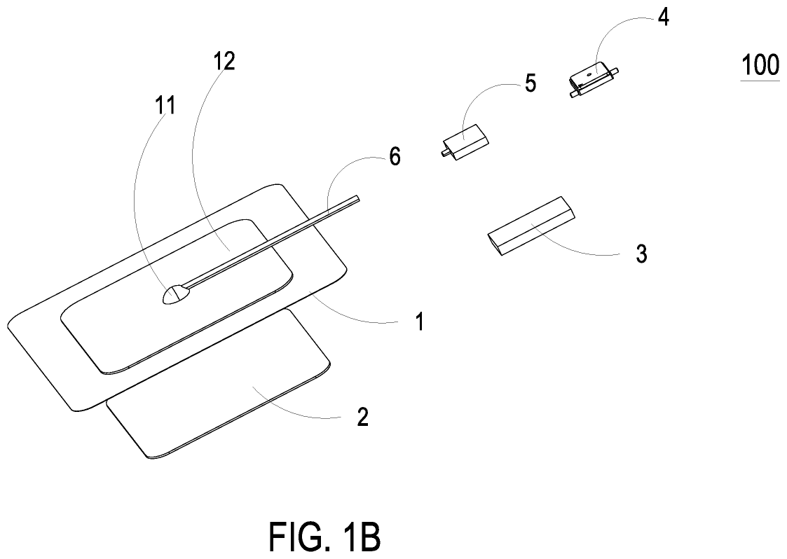

Please refer to FIGS. 1A and 1B, which are the schematic perspective views illustrating the medical negative pressure lamination component 100 of a first embodiment of the present disclosure. As shown in FIGS. 1A and 1B, the medical negative pressure lamination component 100 is used to be attached to a skin surface of a user and includes an airtight patch 1, a dressing patch 2, a battery module 3, a slim-type pump 4 and a sensing and controlling module 5. The airtight patch 1 includes a communication portion 11 and a dressing area 12. The communication portion 11 and the dressing area 12 are in fluid communication with each other. The dressing area 12 is configured to accommodate the dressing patch 2. The battery module 3 is used to supply a driving power and is electrically connected to the slim-type pump 4 and the sensing and controlling module 5. The sensing and controlling module 5 is electrically connected to the slim-type pump 4 so as to detect and control a gas pressure and a gas flow provided by the slim-type pump 4 when the slim-type pump 4 is actuated.

In this embodiment, the dressing patch 2 includes a dressing used for wounds so as to be attached to the wounds of the user. In some embodiments, the dressing patch 2 is a medicine patch, such as a sore patch, a motion sickness patch, an angina pectoris patch, an Alzheimer's disease patch, a Parkinson's disease patch, an Analgesic patch for cancer, a post-herpes neuralgia patch or other prescription patches which can be utilized on the skin surface of the user. The dressing patch 2 is attached to the skin surface where has wounds or is the affected area such as chest, back, arm, abdomen, buttock, thigh, etc., and then the airtight patch 1 covers on the dressing patch 2, and accommodates the dressing patch 2 in the dressing area 12. Thereby, both of the airtight patch 1 and the dressing patch 2 are attached to the skin surface of the user. Thereafter, the slim-type pump 4 is actuated, and the air between the airtight patch 1 and the skin surface is drawn out by the slim-type pump 4 through the communication portion 11. As a result, a negative pressure is formed between the airtight patch 1 and the skin surface, and the airtight patch 1 and the dressing patch 2 are tightly attached to the skin surface of the user.

In addition, in this embodiment, the medical negative pressure lamination component 100 further includes a connection hose 6. The connection hose 6 is connected between the communication portion 11 of the airtight patch 1 and the sensing and controlling module 5.

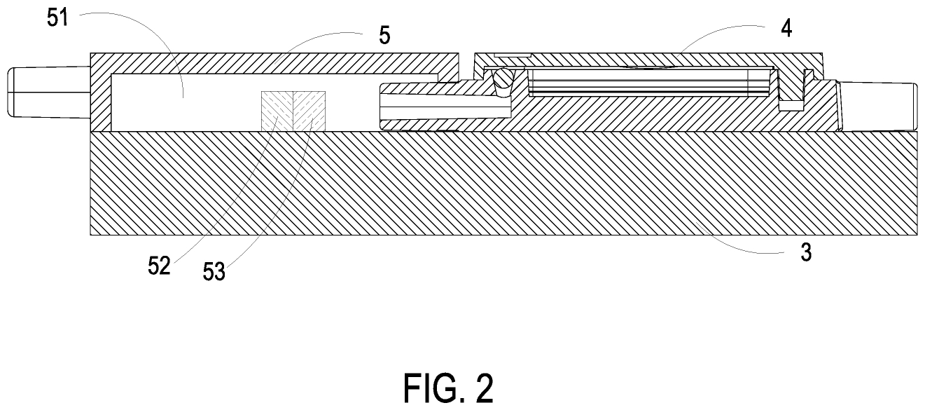

Please refer to FIG. 2. The sensing and controlling module 5 includes a gas passage 51, a sensor 52 and a controller 53. The gas passage 51 is connected between and in fluid communication with the connection hose 6 and the slim-type pump 4. The sensor 52 and the controller 53 are located in the gas passage 51. When the slim-type pump 4 is actuated, the air between the airtight patch 1 and the skin surface of the user is drawn out by the slim-type pump 4 and passes through the communication portion 11, the connection hose 6 and the sensor 52, and the gas flow and the gas pressure in the gas passage 51 is detected by the sensor 52. The controller 53 is electrically connected to the sensor 52 and the slim-type pump 4 and controls and adjusts the operation of the slim-type pump 4 in accordance with the gas flow and the gas pressure detected by the sensor 52.

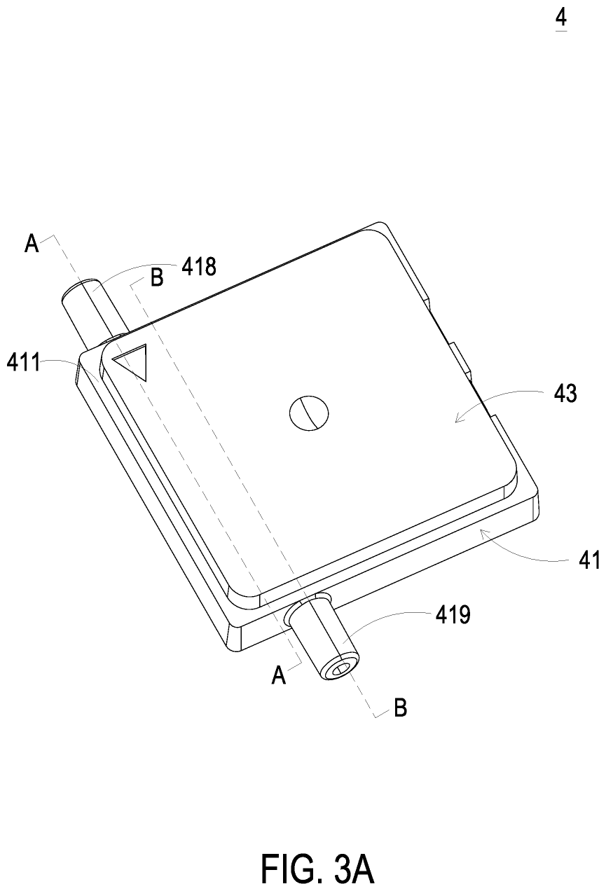

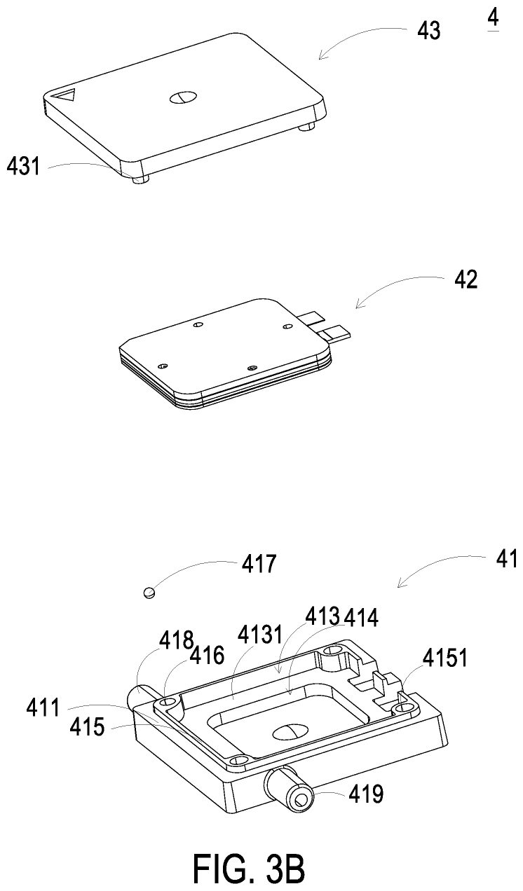

Please refer to FIGS. 3A and 3B. The slim-type pump 4 includes a base plate 41, a gas pump 42 and a top covering 43. The gas pump 42 is accommodated in the base plate 41, and then the top covering 43 is fixed on the base plate 41.

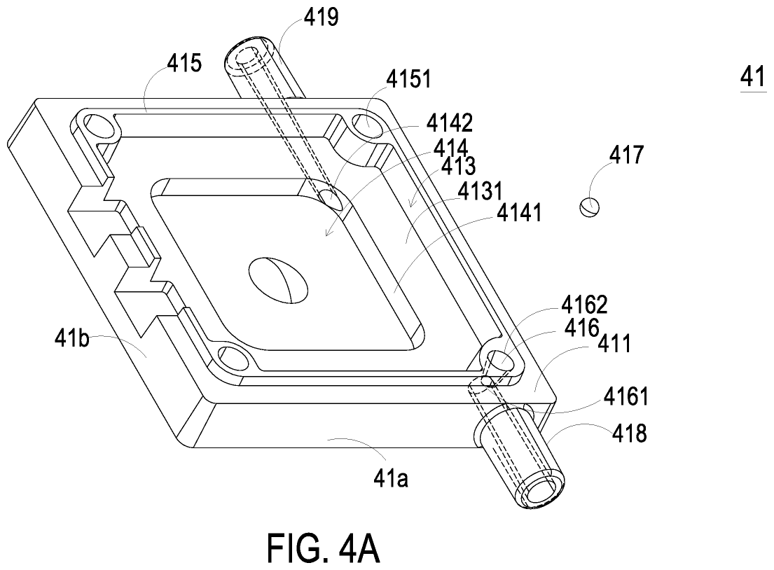

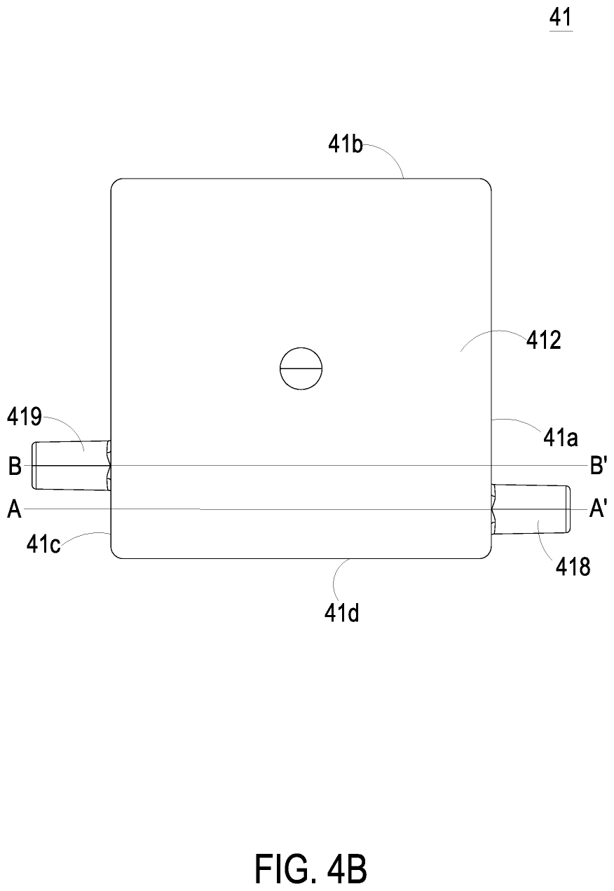

Please refer to FIGS. 4A and 4B. The base plate 41 includes a first surface 411, a second surface 412, an accommodation groove 413, an outlet groove 414, a positioning portion 415, a ventilating hole 416, a closure ball 417, an inlet tube 418, an outlet tube 419, a first sidewall 41a, a second sidewall 41b, a third sidewall 41c and a fourth sidewall 41d. The first surface 411 and the second surface 412 are two surfaces opposite to each other. The accommodation groove 413 is recessed from the first surface 411 and includes an accommodation surface 4131. The outlet groove 414 is recessed from the accommodation surface 4131 and includes a lateral wall 4141 and an outlet channel 4142. The outlet channel 4142 is located on the lateral wall 4141. The positioning portion 415 in square shape is protruded from the first surface 41 and surrounds the accommodation groove 413. The ventilating hole 416 is located on the positioning portion 415 and includes an inlet end 4161 and a ventilating end 4162. The ventilating end 4162 is in fluid communication with the accommodation groove 413. The inlet tube 418 is extended outwardly from the first sidewall 41a and is in fluid communication with the inlet end 4161 of the ventilating hole 416. The outlet tube 419 is extended outwardly from the third sidewall 41c opposite to the first sidewall 41a and is in fluid communication with the outlet channel 4142 of the outlet groove 414. The inlet tube 418 and the outlet tube 419 are spatially misaligned with each other. Notably, the inlet tube 418 and the outlet tube 419 can be disposed on the second sidewall 41b or the fourth sidewall 41d, but not limited thereto.

In this embodiment, the closure ball 417 is spherical, and the ventilating hole 416 has a circular profile. A diameter of the closure ball 417 is ranged between a diameter of the ventilating end 4162 and a diameter of the inlet end 4161 of the ventilating hole 416. More specifically, the diameter of the closure ball 417 is ranged between 0.5 mm and 1 mm. In some embodiments, the diameter of the closure ball 417 is 0.8 mm, and the closure ball 417 is a steel ball.

In this embodiment, the ventilating hole 416 is in a tapered cone profile and is gradually shrunk from the ventilating end 4162 to the inlet end 4161. The closure ball 417 is accommodated in the ventilating hole 416. A slope angle of the cone profile of the ventilating hole 416 is ranged between 10 degrees and 14 degrees. In one embodiment, the slope angle is 12 degrees. The diameter of the inlet end 4161 is 0.68 mm, and the diameter of the ventilating end 4162 is 1.2 mm.

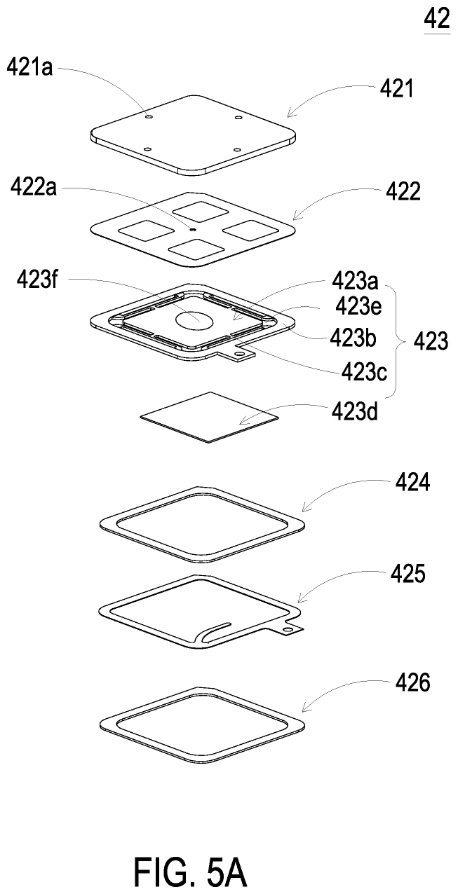

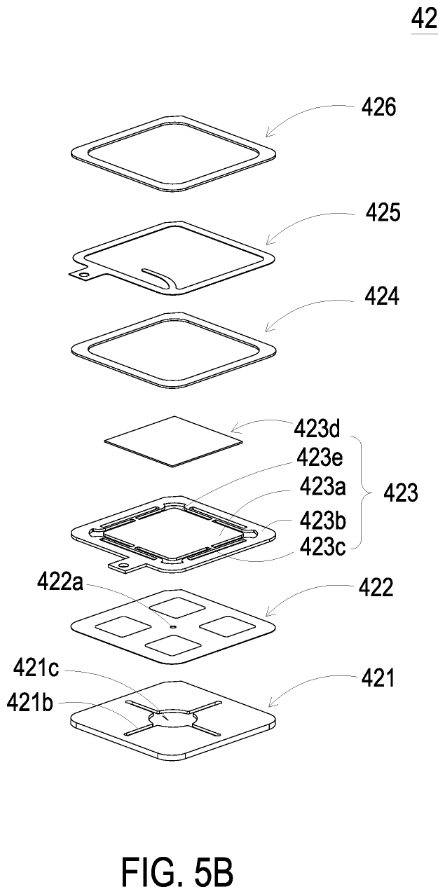

In this embodiment, the gas pump 42 is disposed on the accommodation surface 4131 of the accommodation groove 413 and covers the outlet groove 414. Please refer to FIGS. 5A and 5B. In the embodiment, the gas pump 42 includes a gas inlet plate 421, a resonance plate 422, a piezoelectric actuator 423, a first insulation plate 424, a conducting plate 425 and a second insulation plate 426, which are stacked on each other sequentially. In the embodiment, the gas inlet plate 421 includes at least one inlet aperture 421a, at least one convergence channel 421b and a convergence chamber 421c. The at least one gas inlet aperture 421a is disposed to inhale the gas. The at least one gas inlet aperture 421a correspondingly penetrates through the gas inlet plate 421 into the at least one convergence channel 421b, and the at least one convergence channel 421b is converged into the convergence chamber 421c. Therefore, the gas inhaled through the at least one gas inlet aperture 421a is converged into the convergence chamber 421c. The number of the gas inlet apertures 421a is the same as the number of the convergence channels 421b. In the embodiment, the numbers of the gas inlet apertures 421a and the convergence channels 421b are exemplified by four, respectively, but not limited thereto. The four gas inlet apertures 421a penetrate through the gas inlet plate 421 into the four convergence channels 421b respectively, and the four convergence channels 421b converge to the convergence chamber 421c.

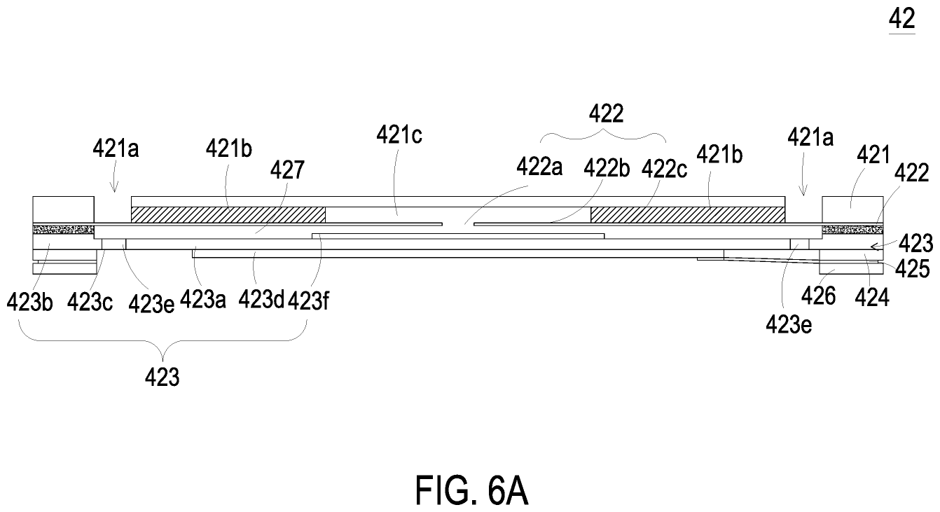

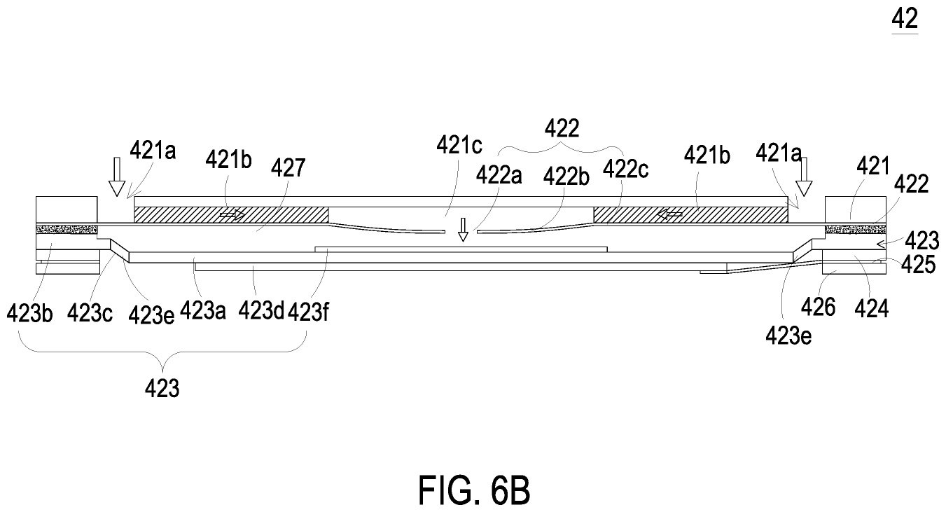

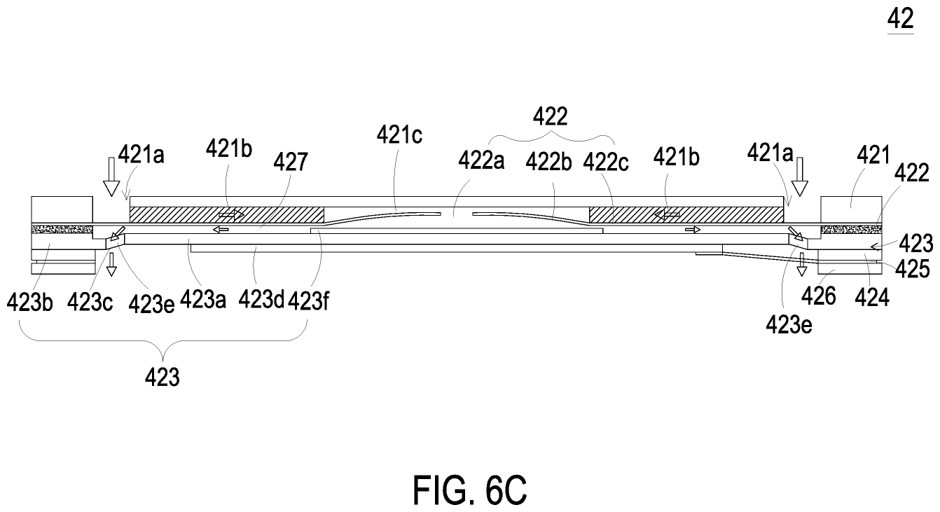

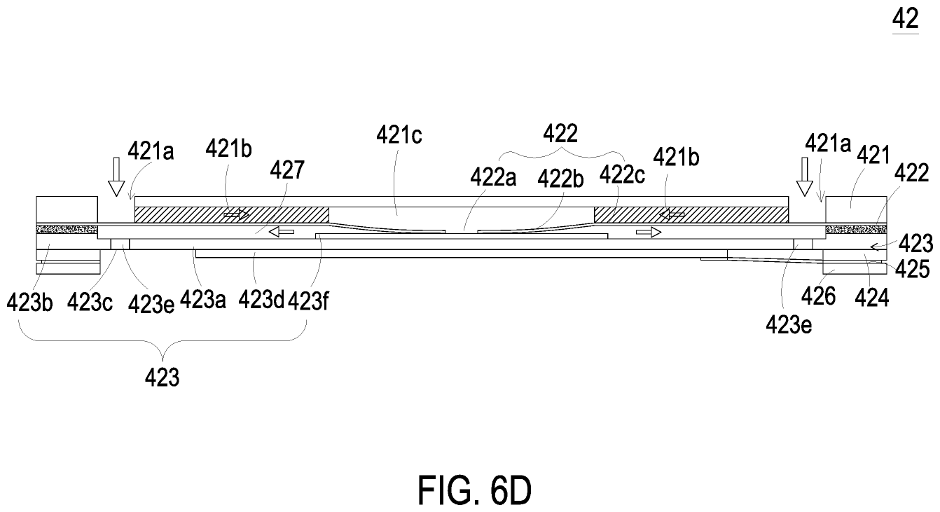

Please refer to FIGS. 5A, 5B and 6A. The resonance plate 422 is attached to the gas inlet plate 421. The resonance plate 422 has a central aperture 422a, a movable part 422b and a fixed part 422c. The central aperture 422a is located at a center of the resonance plate 422 and is corresponding to the convergence chamber 421c of the gas inlet plate 421. The movable part 422b surrounds the central aperture 422a and is corresponding to the convergence chamber 421c. The fixed part 422c is disposed around the periphery of the resonance plate 422 and securely attached on the gas inlet plate 421.

Please refer to FIGS. 5A, 5B and 6A, again. The piezoelectric actuator 423 is attached to the resonance plate 422 and is corresponding in position to the resonance plate 422. The piezoelectric actuator 423 includes a suspension plate 423a, an outer frame 423b, at least one bracket 423c, a piezoelectric element 423d, at least one clearance 423e and a bulge 423E The suspension plate 423a is square-shaped because the square suspension plate 423a is more power-saving than the circular suspension plate. Generally, the consumed power of the capacitive load operated at the resonance frequency would induce as the resonance frequency raised. Since the resonance frequency of the square suspension plate 423a is obviously lower than that of the circular square suspension plate, the consumed power of the square suspension plate 423a would be lesser. Therefore, the square suspension plate 423a in the embodiment has the advantage of power-saving. In the embodiment, the outer frame 423b is disposed around the periphery of the suspension plate 423a, and at least one bracket 423c is connected between the suspension plate 423a and the outer frame 423b for elastically supporting the suspension plate 423a. The piezoelectric element 423d has a side, and the length of the side of the piezoelectric element 423d is less than or equal to that of the suspension plate 423a. The piezoelectric element 423d is attached to a surface of the suspension plate 423a. When a voltage is applied to the piezoelectric element 423d, the suspension plate 423a is driven to undergo the bending vibration. The at least one clearance 423e is formed between the suspension plate 423a, the outer frame 423b and the at least one bracket 423c for allowing the gas to flow through. The bulge 423f is formed on a surface of the suspension plate 423a opposite to the surface of the suspension plate 423a attached to the piezoelectric element 423d. In the embodiment, the bulge 423f is formed by using an etching process on the suspension plate 423a. Accordingly, the bulge 423f of the suspension plate 423a is integrally formed and protrudes from the surface opposite to that attached on the piezoelectric element 423d, and formed a convex structure.

Please refer to FIGS. 5A, 5B and 6A. In the embodiment, the gas inlet plate 421, the resonance plate 422, the piezoelectric actuator 423, the first insulation plate 424, the conducting plate 425 and the second insulation plate 426 are stacked and assembled sequentially. A chamber space 427 is formed between the suspension plate 423a and the resonance plate 422, and the chamber space 427 is formed by filling a gap between the resonance plate 422 and the outer frame 423b of the piezoelectric actuator 423 with a material, such as a conductive adhesive, but not limited thereto. Therefore, a specific depth between the resonance plate 422 and the suspension plate 423a is maintained and formed as the chamber space 427. So as to guide the gas to pass rapidly. In addition, since the resonance plate 422 and the suspension plate 423a are maintained at a suitable distance, the contact interference therebetween can be reduced, thereby largely reducing the noise. In other embodiments, the thickness of the conductive adhesive filled into the gap between the resonance plate 422 and the outer frame 423b of the piezoelectric actuator 423 can be reduced by increasing the height of the outer frame 423b of the piezoelectric actuator 423. Therefore, the entire assembling structure of gas pump 42 would not be indirectly influenced by the hot-pressing temperature and the cooling temperature, and avoiding the actual distance between the suspension plate 423a and the resonance plate 422 of the chamber space 427 being affected by the thermal expansion and contraction of the filling material of the conductive adhesive, but is not limited thereto. In addition, since the transportation effect of the gas pump 42 is affected by the chamber space 427, it is very important to maintain a constant chamber space 427, so as to provide a stable transportation efficiency of the gas pump 42.

In order to understand the actuation steps of the gas pump 42, please refer to FIGS. 6B to 6D. Referring to FIG. 6B, first, when the piezoelectric element 423d of the piezoelectric actuator 423 is deformed in response to an applied voltage, the suspension plate 423a is driven to displace in the direction away from the resonance plate 422. In that, the volume of the chamber space 427 is increased, a negative pressure is formed in the chamber space 427, and the gas in the convergence chamber 421c is introduced into the chamber space 427. At the same time, the resonance plate 422 is in resonance and is thus displaced synchronously, and thereby, increased the volume of the convergence chamber 421c. Since the gas in the convergence chamber 421c is introduced into the chamber space 427, the convergence chamber 421c is also result in a negative pressure state, and the gas is inhaled into the convergence chamber 421c through the gas inlet apertures 421a and the convergence channels 421b. Then, as shown in FIG. 6C, the piezoelectric element 423d drives the suspension plate 423a to displace toward the resonance plate 422 to compress the chamber space 427. Similarly, the resonance plate 422 is actuated and displaced away from the suspension plate 423a in resonance to the suspension plate 423a, and compress the air in the chamber space 427. Thus, the gas in the chamber space 427 is further transmitted downwardly to pass through the clearances 423e and achieves the effect of gas transportation. Finally, as shown in FIG. 6D, when the suspension plate 423a resiliently moves back to an initial state, the resonance plate 422 displaces toward the suspension plate 423a due to its inertia momentum, and keeps on pushing the gas in the chamber space 427 toward the clearances 423e. Meanwhile, the volume of the convergence chamber 421c is increased. Thus, the gas outside is continuously inhaled and passed through the gas inlet apertures 421a and the convergence channels 421b, and converged in the convergence chamber 421c. By repeating the actuations illustrated in FIGS. 6B to 6D continuously, the gas pump 42 can continuously transport the gas at high speed. The gas enters the gas inlet apertures 421a, flows through a flow path formed by the gas inlet plate 421 and the resonance plate 422 and result in a pressure gradient, and then transported through the clearances 423e, so as to achieve the operation of gas transporting of the gas pump 42.

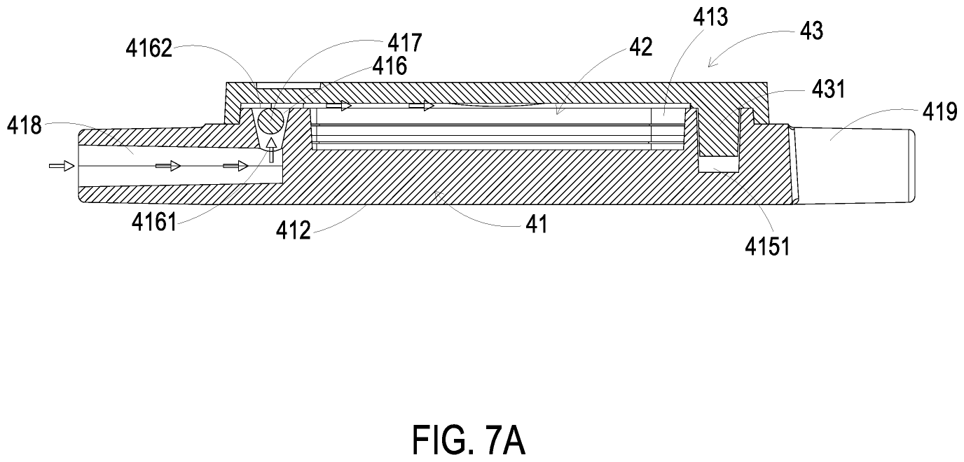

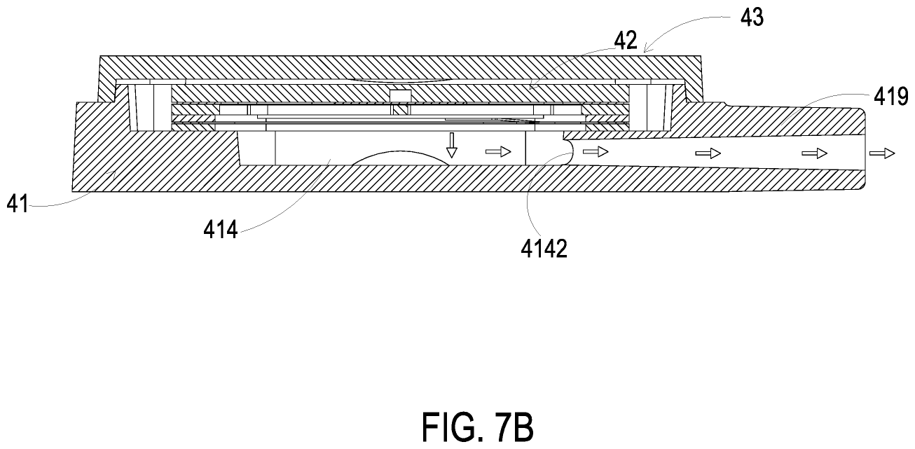

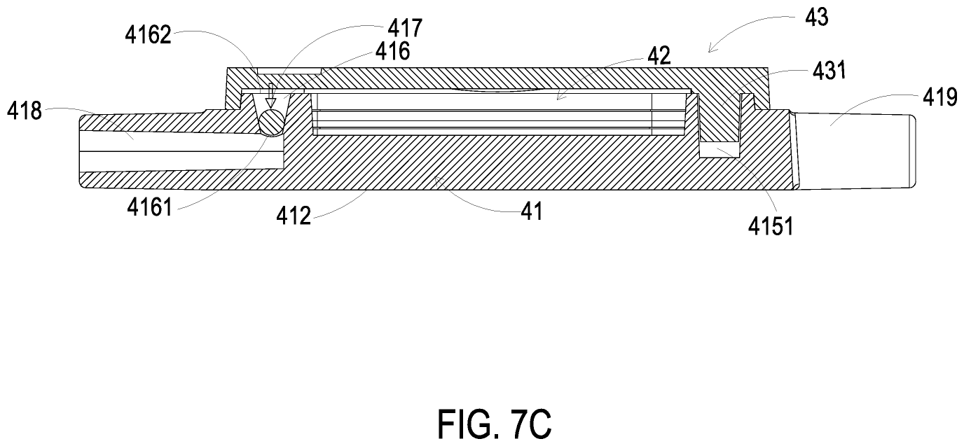

FIG. 7A is a schematic cross-section view taken along a section line A-A′ of FIG. 3A. FIG. 7B is a schematic cross-section view taken along a section line B-B′ of FIG. 3A. Please refer to FIG. 7A, when the gas pump 42 is actuated, the gas inside the accommodation groove 413 is drawn and transported downwardly to the outlet groove 414 (as shown in FIG. 7B). As a result, the space of the accommodation groove 413 is in a negative pressure state. Thereafter, the gas outside the slim-type pump 4 enters the slim-type pump 4 through the inlet tube 418 of the base plate 41 and pushes the closure ball 417 inside the ventilating hole 416 upwardly. Thereby, the closure ball 417 is leaved from the inlet end 4161 of the ventilating hole 416, and the gas flows from the inlet tube 418 to the ventilating hole 416 through the inlet end 4161. Since the diameter of the ventilating end 4162 is greater than the diameter of the closure ball 417, the closure ball 417 is not capable of sealing the ventilating end 4162. The gas flows to the accommodation groove 413 through the ventilating end 4162 and then flows toward the outlet groove 414. Please refer to FIG. 7B. When the gas is transported to the outlet groove 414, the gas is then transported to the outlet tube 419 through the outlet channel 4142 and is discharged from the outlet tube 419. Thereby, the gas transportation process is completed.

Please refer to FIG. 7C. When the gas pump 42 is disabled, the gas pressure in the accommodation groove 413 is higher than the gas pressure outside the slim-type pump 4. Consequently, the gas inside the accommodation groove 413 flows to the ventilating hole 416 and pushes the closure ball 417 located on the ventilating end 4162 to the inlet end 4161. Since the diameter of the closure ball 417 is greater than the diameter of the inlet end 4161, the inlet end 4161 is sealed by the closure ball 417 when the closure ball 417 is pushed to the inlet end 4161. As a result, the gas is prevented from passing through the inlet end 4161, and the effect of avoiding the gas reflowing can be achieved.

Please refer to FIGS. 3B and 7C. The positioning portion 415 of the base plate 41 includes at least one fixing hole 4151. In this embodiment, the positioning portion 415 includes three fixing holes 4151, but not limited thereto. The top covering 43 includes at least one fixing column 431. The number and the position of the fixing column 431 are corresponding to those of the fixing hole 4151. The fixing columns 431 respectively pass through the corresponding fixing holes 4151 for positioning and fixing. Thereby, the top covering 43 is firmly disposed on the positioning portion 415 and covers the accommodation groove 413.

Based on the above descriptions, the present disclosure provides a medical negative pressure lamination component. Through drawing out the gas between the airtight patch and the slim-type pump by the slim-type pump, the negative pressure can be formed therebetween, and the effect of tight attachment can be achieved. Moreover, through using the sensing and controlling module to adjust and control the gas pressure and the gas flow provided by the slim-type pump, the medical negative pressure lamination component is adaptable to various skin surfaces of the different users. The pressure between the airtight patch and the skin surface is also adjustable so as to provide comfort. Thereafter, by introducing the gas into the airtight patch through the connection hose, the gas pressures inside and outside the medical negative pressure lamination component will become balanced, and the airtight patch can be removed from the skin surface. That is, the airtight patch can be easily removed without hurting the user, and no glue is remained on the skin surface.

While the disclosure has been described in terms of what is presently considered to be the most practical and preferred embodiments, it is to be understood that the disclosure needs not be limited to the disclosed embodiment.

What is claimed is:

1. A medical negative pressure lamination component for attaching to a skin surface, the medical negative pressure lamination component comprising:
an airtight patch comprising a communication portion and a dressing area, wherein the communication portion and the dressing area are in fluid communication with each other;
a dressing patch accommodated in the dressing area;
a battery module;
a slim-type pump electrically connected to the battery module; and
a sensing and controlling module electrically connected to the battery module and the slim-type pump for detecting and controlling a gas pressure and a gas flow provided by the slim-type pump,
wherein the dressing patch is attached on the skin surface, covered by the airtight patch, and then accommodated in the dressing area, wherein when the slim-type pump is actuated, the air between the airtight patch and the skin surface is drawn out by the slim-type pump through the communication portion, and a negative pressure is formed between the airtight patch and the skin surface, so that the airtight patch and the dressing patch are tightly attached to the skin surface.

2. The medical negative pressure lamination component according to claim 1, further comprising a connection hose connected between the communication portion of the airtight patch and the sensing and controlling module.

3. The medical negative pressure lamination component according to claim 2, wherein the sensing and controlling module comprises a gas passage, a sensor and a controller, wherein the gas passage is connected between and in fluid communication with the connection hose and the slim-type pump, the sensor is disposed in the gas passage to detect the gas flow and the gas pressure in the gas passage, and the controller is electrically connected to the sensor and the slim-type pump to control and adjust the operation of the slim-type pump according to the gas flow and the gas pressure detected by the sensor.

4. The medical negative pressure lamination component according to claim 1, wherein the slim-type pump comprises:
a base plate comprising:
a first surface;
a second surface opposite to the first surface;
an accommodation groove recessed from the first surface and comprising an accommodation surface;
an outlet groove recessed from the accommodation surface and comprising an outlet channel;
a positioning portion protruded from the first surface and surrounding the accommodation groove;
a ventilating hole located on the positioning portion and comprising an inlet end and a ventilating end, wherein the ventilating end is in fluid communication with the accommodation groove, and the ventilating hole is gradually shrunk from the ventilating end to the inlet end;
a closure ball accommodated in the ventilating hole;
an inlet tube in fluid communication with the inlet end of the ventilating hole; and
an outlet tube in fluid communication with the outlet channel of the outlet groove;
a gas pump disposed on the accommodation surface of the accommodation groove and covering the outlet groove; and
a top covering disposed on the positioning portion and covering the accommodation groove,
wherein a diameter of the closure ball is ranged between a diameter of the ventilating end and a diameter of the inlet end of the ventilating hole.

5. The medical negative pressure lamination component according to claim 4, wherein the diameter of the closure ball is ranged between 0.5 mm and 1 mm.

6. The medical negative pressure lamination component according to claim 5, wherein the diameter of the closure ball is 0.8 mm.

7. The medical negative pressure lamination component according to claim 4, wherein the closure ball is a steel ball.

8. The medical negative pressure lamination component according to claim 4, wherein the positioning portion comprises at least one fixing hole, and the top covering comprises at least one fixing column passing through the at least one fixing hole.

9. The medical negative pressure lamination component according to claim 4, wherein the gas pump comprises:
a gas inlet plate having at least one gas inlet aperture, at least one convergence channel and a convergence chamber, wherein the at least one gas inlet aperture is disposed to inhale the gas, the at least one gas inlet aperture correspondingly penetrates through the gas inlet plate and in fluid communication with the at least one convergence channel, and the at least one convergence channel is converged into the convergence chamber, so that the gas inhaled through the at least one gas inlet aperture is converged into the convergence chamber;
a resonance plate disposed on the gas inlet plate and having a central aperture, a movable part and a fixed part, wherein the central aperture is disposed at a center of the resonance plate and corresponds to the center of the convergence chamber of the gas inlet plate, the movable part surrounds the central aperture and corresponds to the convergence chamber, and the fixed part surrounds the movable part and is fixedly attached on the gas inlet plate; and
a piezoelectric actuator correspondingly disposed on the resonance plate;
wherein a chamber space is formed between the resonance plate and the piezoelectric actuator, so that when the piezoelectric actuator is driven, the gas introduced from the at least one gas inlet aperture of the gas inlet plate is converged to the convergence chamber through the at least one convergence channel, and flows through the central aperture of the resonance plate so as to produce a resonance effect with the movable part of the resonance plate and the piezoelectric actuator to transport the gas.

10. The medical negative pressure lamination component according to claim 9, wherein the piezoelectric actuator comprises:
a suspension plate in square-shaped permitted to undergo a bending vibration;
an outer frame surrounding the suspension plate;
at least one bracket connected between the suspension plate and the outer frame to provide an elastic support for the suspension plate; and
a piezoelectric element having a side, wherein a length of the side of the piezoelectric element is less than or equal to that of the suspension plate, and the piezoelectric element is attached on a surface of the suspension plate, wherein when a voltage is applied to the piezoelectric element, the suspension plate is driven to undergo the bending vibration.

11. The medical negative pressure lamination component according to claim 10, wherein the gas pump further comprises a first insulation plate, a conducting plate and a second insulation plate, and the gas inlet plate, the resonance plate, the piezoelectric actuator, the first insulation plate, the conducting plate and the second insulation plate are stacked and assembled sequentially.
